(19) United States
(12) Patent Application Publication
(10) Pub. No.: US 2023/0270642 A1
(43) Pub. Date: Aug. 31, 2023

(54) AQUEOUS COMPOSITION FOR CARING FOR AND/OR MAKING UP KERATIN MATERIALS COMPRISING A FATTY ACID MONOESTER, A FATTY ACID SALT, A PULLULAN AND A POLYOL

(71) Applicant: L'OREAL, Paris (FR)

(72) Inventors: Bouchra BOUARFA, Chevilly La Rue (FR); Mohamed BOULARAS, Chevilly La Rue (FR); Sophie BEAUMARD, Chevilly La Rue (FR); Delphine LAURENT, Chevilly La Rue (FR)

(21) Appl. No.: 18/009,003

(22) PCT Filed: Jun. 4, 2021
(86) PCT No.: PCT/EP2021/065007

§ 371 Date: Dec. 8, 2022

(30) Foreign Application Priority Data
Jun. 8, 2020 (FR) 2005956

(51) Int. Cl. A61K 8/37; A61Q 1/10

(52) U.S. Cl. CPC A61K 8/37; A61Q 1/10; A61K 2800/87; A61K 2800/5922

(57) ABSTRACT

Aqueous composition for caring for and/or making up keratin materials comprising a fatty acid monoester, a fatty acid salt, a pullulan and a polyol

The present application relates to a composition comprising, notably in a physiologically acceptable medium:
(1) at least one linear fatty acid monoester of formula (I) below:

R₁—O—R₂ (I)
wherein R₁ and R₂ are linear and saturated and have, independently of one another, a number of carbon atoms greater than or equal to 20, with R₁ representing an acyl radical, and R₂ representing an alkyl radical, and
(2) at least one fatty acid having from 14 carbon atoms to less than 20 carbon atoms, and
(3) at least one base capable of at least partially neutralizing said fatty acid, and
(4) at least one pullulan, and
(5) at least one polyol, and
(6) water; said composition containing less than 5.0% by weight of wax(es).

The invention also relates to a process for coating keratin materials, in particular the skin, notably the contour of the eyes, eyelashes or eyebrows, and keratin fibers, such as the eyelashes and/or the eyebrows, comprising a step of applying to said keratin materials at least one composition as defined above.

### TECHNICAL FIELD

The present invention relates to the field of caring for and/or making up keratin materials, and is directed towards proposing compositions more particularly intended for making up the eyelashes or the eyebrows.

In general, compositions intended for making up keratin fibers, for example the eyelashes, aim to density the thickness and the visual perception of the eyelashes and ultimately the gaze. These mascaras are described as aqueous or cream mascaras when they are formulated in an aqueous base, and anhydrous mascaras when they are formulated as a dispersion in an organic solvent medium.

A great diversity of cosmetic effects may be afforded by applying a mascara to keratin fibers and notably the eyelashes, for instance a volumizing, lengthening, thickening and more particularly charging makeup effect.

These effects are mostly adjusted through the amount and nature of the particles and most particularly those of the waxes present in the mascaras. In general, mascaras in fact have a significant amount of wax(es) and notably from 10% to 35% by weight of waxes, more generally from 15% to 30% by weight, relative to the total weight thereof.

For obvious reasons, improving the textures of mascara which condition the manifestation of one or more makeup effects is a constant preoccupation of cosmetic formulators.

Moreover, the specific effects associated with a particular formulation, for example charging and moreover providing excellent separation of the made-up eyelashes, are expected to be reproduced virtually identically by all the production batches of one and the same formulation.

In order to meet these expectations and/or objectives, it is therefore necessary to be capable of precisely adjusting the texture of a mascara and of reproducing it as faithfully as possible with batches that are not necessarily produced at the same time but which are identical in terms of ingredients and must therefore provide makeup effects that are in theory also identical.

### PRIOR ART

However, as specified above, most of the mascaras currently available are formulated with a significant amount of waxes. In point of fact, as detailed in the document Ullmann's Encyclopedia of Industrial Chemistry 2015, Wiley-VCH Verlag GmbH & Co. KGaA, Weinheim 10.1002/14356007.a28.pub2, most waxes are not constituted of a single chemical compound, but are instead complex mixtures. They may be mixtures of oligomers and/or of polymers which, in many cases, also have varied molar masses, varied molar mass distributions and also varied degrees of branching. Thus, a polar wax is conventionally made up of a mixture of alkanes, fatty alcohols and fatty esters, the fatty-chain length of which varies according to the melting point.

It is therefore very difficult for the producers of these waxes to guarantee a rigorously identical composition for all production batches. More specifically, there may be, between several production batches of one and the same wax, a variability in terms of the chemical nature of some of its constituent compounds. Likewise, the proportionality of some of its constituent compounds may also vary between production batches.

For obvious reasons, these variabilities have a not insignificant impact on the properties of the wax and therefore on those of the mascara incorporating this wax in significant amount. Thus, two mascara formulations of identical composition and therefore produced from one and the same conventional wax and in the same amount, can nevertheless diverge in terms of rheological properties and therefore of texture and mechanical properties of the deposition of the product on keratin fibers, such as resistance to flaking, if they were produced from two separate production batches of this wax.

Consequently, the use of conventional waxes, in particular in significant amount, in mascara compositions does not make it possible to guarantee for users the reproduction of the finely adjusted and totally identical rheological properties and mechanical properties of the deposition of the product on keratin fibers, such as resistance to flaking, in all the mascara specimens of one and the same composition.

The present invention aims precisely to provide mascara compositions which have overcome this limitation.

Thus, a first objective of the present invention is to obtain mascara compositions of which the texturing properties are finely adjustable and reproducible.

A second objective of the present invention is to obtain mascara compositions of which the mechanical properties of the deposition of the product on keratin fibers, such as resistance to flaking, are finely adjustable and reproducible.

Another objective of the present invention is to provide a mascara architecture produced using a predominant weight proportion of single-component ingredients. The predominant use of single-component ingredients advantageously makes it possible to eliminate the risk of composition variability that may exist between several production batches of a multicomponent ingredient and therefore to eliminate the risk of its impact on the final properties of the mascara.

Another objective of the present invention is to provide a mascara architecture which makes it possible to significantly reduce or even dispense with the use of waxes, but which nevertheless remains very satisfactory in terms of makeup effect.

Unexpectedly, the inventors have found that it is possible to significantly or even totally dispense with waxes as texturing agent in formulations for making up and/or caring for keratin fibers and therefore with the limitations mentioned above, on condition that very specific compounds are combined in such formulations.

In the course of its research, the applicant has discovered, unexpectedly, that the objectives as defined above are achieved by using a composition comprising, notably in a physiologically acceptable medium:
(1) at least one linear fatty acid monoester of formula (I):
[Chem 1]

R₁—O—R₂ (I)
wherein R₁ and R₂ are linear and saturated and have, independently of one another, a number of carbon atoms greater than or equal to 20, with R₁ representing an acyl radical, and R₂ representing an alkyl radical, and
(2) at least one fatty acid having from 14 carbon atoms to less than 20 carbon atoms, and
(3) at least one base capable of at least partially neutralizing said fatty acid, and
(4) at least one pullulan, and
(5) at least one polyol, and
(6) water; said composition containing less than 5.0% by weight of wax(es).

Unexpectedly, the inventors have in fact noted that the formulation comprising at least one linear fatty acid monoester (1), at least one fatty acid having from 14 carbon atoms to less than 20 carbon atoms (2) in a form neutralized by a base (3), at least one pullulan (4), at least one polyol (5) and water (6) makes it possible to obtain compositions of which the texture and the mechanical properties of the deposition of the formulation on keratin fibers can be finely adjusted and guaranteed in terms of reproducibility.

As emerges from what follows, these new compositions are advantageous in several respects.

First of all, compounds (1) to (6) required according to the invention are as an individualized compound, a single-component compound or a compound with a well-defined number of components, as opposed to the majority of conventional waxes which are often multicomponent or even have an indefinite number of compounds, such as natural waxes and some synthetic waxes.

These two specificities are particularly advantageous since they make it possible to eliminate a risk of variability with regard to their respective compositions.

As emerges from the examples below, the compositions in accordance with the invention and based on the use of compounds (1) as texturing agent prove to be very satisfactory in terms of makeup effects.

Thus, compositions according to the invention may have a creamy texture which proves to be finely adjustable by virtue of the use of the required combination according to the invention.

The obtention of these properties is conditioned by the use of the compounds (1) to (6) and advantageously does not therefore require the additional presence of waxes, in particular in significant amount.

Thus, the compositions according to the invention advantageously comprise less than 5.0% of waxes as defined below.

The term “waxes” refers to lipophilic compounds, which are solid at ambient temperature (25° C.) and at atmospheric pressure (760 mmHg), with a reversible solid/liquid change of state, which have a melting point of greater than or equal to 40° C., which may be up to 120° C.

For the purposes of the invention, the waxes to which this abovementioned amount limitation relates are distinct from those capable of being embodied by the component which is a monoester of fatty acid(s) (1) required according to the invention and additives of the fatty alcohol type.

This discovery forms the basis of the invention.

### SUBJECTS OF THE INVENTION

Thus, according to one of its aspects, the present invention relates to a composition comprising, notably in a physiologically acceptable medium:
(1) at least one linear fatty acid monoester of formula (I):

R₁—O—R₂ (I)
wherein R₁ and R₂ are linear and saturated and have, independently of one another, a number of carbon atoms greater than or equal to 20, with R₁ representing an acyl radical, and R₂ representing an alkyl radical, and
(2) at least one fatty acid having from 14 carbon atoms to less than 20 carbon atoms, and
(3) at least one base capable of at least partially neutralizing at least one fatty acid (2), and
(4) at least one pullulan, and
(5) at least one polyol, and
(6) water; said composition containing less than 5.0% by weight of wax(es).

A second subject of the present invention is a cosmetic process for making up and/or caring for human keratin materials, such as the skin, notably the contour of the eyes, the contour of the eyelashes, the contour of the eyebrows; keratin fibers such as the eyelashes and the eyebrows, consisting in applying to said keratin materials a composition as defined above.

### Definitions

In the context of the present invention, the term “keratin material” is notably intended to mean the skin, notably the contour of the eyes, the contour of the eyelashes, the contour of the eyebrows; keratin fibers such as the eyelashes and the eyebrows. For the purposes of the present invention, this term “keratin fibers” also extends to synthetic false eyelashes.

The term “physiologically acceptable” is intended to mean compatible with the skin and/or its integuments, which has a pleasant color, odor and feel, and which does not cause any unacceptable discomfort (stinging or tautness) liable to discourage the consumer from using this composition.

According to another of its aspects, the present invention relates to a process, notably a cosmetic process for caring for and/or making up keratin materials, in particular the eyelashes and/or the eyebrows, comprising at least one step consisting in applying, to said keratin materials, in particular the eyelashes and/or the eyebrows.

### Fatty Acid Monoester

A composition according to the invention comprises at least one linear fatty acid monoester.

A composition according to the invention may comprise at least 5.0% by weight, preferably at least 6.0% by weight, better still at least 7.0% by weight of linear fatty acid monoester(s), relative to the total weight of the composition.

According to one particularly preferred embodiment of the invention, the fatty acid monoester(s) are present in the composition in a content ranging from 6.0% to 35.0% by weight, preferably from 7.0% to 30.0% in weight, or even preferably from 8.0% to 28.0% by weight, relative to the total weight of the composition.

The linear fatty acid monoester(s) (1) considered according to the invention correspond to formula (I) below:

R₁—O—R₂ (I)
wherein R₁ and R₂ are linear and saturated and have, independently of one another, a number of carbon atoms greater than or equal to 20, with R₁ representing an acyl radical, and R₂ representing an alkyl radical.

This or these fatty acid monoester(s) is used during the preparation of a composition according to the invention, in an individualized form or in the form of a mixture comprising exclusively linear fatty acid monoesters of formula (I).

In a preferred embodiment, the fatty acid monoester(s) have a melting point greater than 50° C.

The melting point may be measured by any known method and in particular using a differential scanning calorimeter (DSC).

According to one preferred embodiment of the invention, the acyl and alkyl radicals representing respectively R₁ and R₂ are chosen in such a way that the compound (I) is solid at a temperature of less than or equal to 30° C.

According to one particularly preferred embodiment of the invention, R₁ and R₂ are, respectively, acyl and alkyl radicals having a number of carbon atoms ranging from 20 to 30, preferably from 20 to 24.

According to a particularly preferred embodiment, R₁ and R₂ are, respectively, acyl and alkyl radicals having the same number of carbon atoms.

In particular, the fatty acid monoester according to the invention is chosen from arachidyl arachidate and behenyl behenate.

According to one particularly preferred embodiment of the invention, the linear fatty acid monoester is a behenyl behenate.

A behenyl behenate that is suitable for use in the composition according to the invention may in particular be Kester Wax K-72® sold by the company Koster Keunen, Dub BB® sold by Stéarineries Dubois, or Dermowax BB® sold by Alzo.

### Ionic Surfactant

As specified above, the fatty acid monoester(s) (1) used according to the invention are combined with at least one ionic surfactant resulting from the neutralization of a fatty acid (2) comprising from 14 to less than 20 carbon atoms by a base (3).

### Fatty Acid

The fatty acid according to the invention comprises from 14 to less than 20 carbon atoms. According to one preferred embodiment of the invention, the fatty acid comprises from 16 to less than 20 carbon atoms. According to one particularly preferred embodiment, the number of carbon atoms ranges from 16 to 18.

In particular, the fatty acid(s) according to the invention are chosen from linear fatty acids, saturated fatty acids and mixtures thereof.

According to one particularly advantageous embodiment of the invention, the fatty acid of the ionic surfactant is linear and saturated.

According to a particular embodiment of the invention, the fatty acid(s) are chosen from palmitic acid, stearic acid and mixtures thereof, and preferably comprise at least stearic acid having the INCI name Stearic Acid.

Thus, according to another embodiment of the invention, the composition uses, as fatty acid (2), a mixture of C₁₆-C₁₈ fatty acids, preferably a mixture of fatty acids having 16 carbon atoms, such as palmitic acid, and of fatty acids having 18 carbon atoms, such as stearic acid.

A preferred stearic acid that is suitable for use in the invention is, for example, Stearic Acid 1850® sold by the company Southern Acids.

A composition according to the invention may comprise at least 3.0% by weight of fatty acid(s) (2) relative to the total weight of the composition.

According to a preferred embodiment of the invention, the fatty acid is present in a content ranging from 3.5% to 20.0% by weight, preferably from 4.0% to 20.0% by weight, better still from 4.5% to 15.0% by weight, even better still from 5.0% to 15.0% by weight of fatty acid (2), in particular of stearic acid, relative to the total weight of the composition.

As mentioned above, this fatty acid is used in an ionic form generated via its interaction with an organic base. This base is used at an amount sufficient to be capable of at least partially neutralizing at least one fatty acid (2).

### Base

The composition according to the invention comprises at least one base. This base may be organic or inorganic.

According to a first variant, the base is at least one organic base.

Preferably, the base of organic origin is chosen from amino acids such as arginine; alkanolamines such as monoethanolamine, diethanolamine, triethanolamine, triisopropanolamine, aminomethylpropanol; primary (poly)hydroxyalkylamines such as 2-amino-2-(hydroxymethyl)propane-1,3-diol (also known as tromethamine) and aminomethylpropanediol; and mixtures thereof.

According to one particular embodiment, the base is a primary (poly)hydroxyalkylamine.

According to another particular embodiment, the base is an amino acid, in particular arginine.

The term “primary (poly)hydroxyalkylamine” in particular means a primary dihydroxyalkylamine, it being understood that the term “primary” refers to a primary amine function, i.e. —NH₂, and the alkyl group being a linear or branched C₁-C₈, preferably branched C₄, hydrocarbon-based chain, such as 1,3-dihydroxy-2-methylpropyl. The primary (poly)hydroxyalkylamine is preferably 1,3-dihydroxy-2-methyl-2-propylamine (also known as aminomethylpropanediol or AMPD).

According to one preferred embodiment of the invention, the base of organic origin is aminomethylpropanediol.

Such an aminomethylpropanediol suitable for use in the invention is for example AMPD Ultra PC® sold by the company Angus (Dow Corning).

According to a second variant, the base is at least one inorganic base.

This inorganic base is chosen from alkaline metal hydroxides and ammonium (NH₄⁺).

Preferably, the inorganic base is chosen from sodium hydroxide and potassium hydroxide, and mixtures thereof.

According to a preferred embodiment of the invention, the inorganic base is sodium hydroxide.

The amount of base is adjusted so as to obtain sufficient neutralization to confer effective ionicity on the associated fatty acid (2).

Preferably, the base is present in an amount sufficient to neutralize some or all of the carboxylic functions of the fatty acid(s) (2) comprising from 14 to less than 20 carbon atoms. In a preferred embodiment, the amount of base is such that it is capable of neutralizing all the acid functions of the fatty acid (2).

For example, the composition according to the invention may comprise at least 0.1% by weight, better still at least 0.15% by weight, of base(s), relative to the total weight of the composition.

According to one preferred embodiment of the invention, the base is present in a content ranging from 0.2% to 3.0% by weight, preferably from 0.3% to 2.0% by weight of base(s), in particular of sodium hydroxide or aminomethylpropanediol, relative to the total weight of the composition according to the invention.

According to a particular embodiment of the invention, the compositions according to the invention contain an ionic surfactant derived from the total neutralization of stearic acid with sodium hydroxide.

According to another particular embodiment of the invention, the compositions according to the invention contain an ionic surfactant derived from the total neutralization of stearic acid with aminomethylpropanediol.

The fatty acid (2) and the base (3) making up the neutralized ionic surfactant according to the invention may be introduced into the composition in the form of one and the same commercial material, or one after the other in the form of two distinct commercial materials. Preferably, the fatty acid (2) and the base (3) will be introduced into the composition in the form of two distinct commercial materials.

### Pullulan

The pullulan in accordance with the invention is a polysaccharide constituted of maltotriose units, known under the name α-(1,4)-α(1,6)-glucan. Three glucose units in maltotriose are connected via an α-(1,4) glycoside bond, whereas the consecutive maltotriose units are connected to each other via an α-(1,6) glycoside bond. It is produced from starch by the fungus Aureobasidium pullulans. The pullulan is for example produced under the trade reference Pullulan PF 20® by the Hayashibara group in Japan or under the reference Aqua Beta® by the company Daiso Co. Ltd.

According to one particularly preferred embodiment of the invention, the pullulan is present in the composition in a content ranging from 1% to 7% by weight, preferably from 1% to 6% in weight, or even preferably from 2% to 5% by weight, relative to the total weight of the composition.

### Polyol

For the purposes of the present invention, the term “polyol” should be understood as meaning any organic molecule comprising at least two free hydroxyl groups.

Preferably, a polyol in accordance with the present invention is present in a form that is liquid at ambient temperature (25° C.) and atmospheric pressure (760 mmHg).

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those preferably having 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol and glycerol, and mixtures thereof.

According to one preferred embodiment of the invention, said polyol is chosen from 1,3-propanediol, glycerol and pentylene glycol, and mixtures thereof.

According to one particular embodiment, the composition of the invention comprises 1,3-propanediol and/or glycerol, and more preferentially a mixture of 1,3-propanediol and glycerol.

The polyol(s) are preferably present in the composition in a total content ranging from 5% to 10%, better still from 5% to 7.5% by weight, relative to the total weight of said composition.

According to one preferential embodiment of the invention, the glycerol is used in a maximum amount of 3% by weight relative to the total weight of said composition.

### Water

A composition according to the invention comprises water.

In particular, a composition according to the invention comprises at least 30.0% by weight, better still at least 40.0% by weight, or even a content of from 50% to 60% by weight of water, relative to the total weight of the composition.

In one preferred embodiment of the invention, the composition according to the invention includes:
(1) at least one linear fatty acid monoester of formula (I):

R₁—O—R₂ (I)

wherein R₁ and R₂ are linear and saturated and have, independently of one another, a number of carbon atoms ranging from 20 to 24, with R₁ representing an acyl radical, and R₂ representing an alkyl radical, and
(2) at least one fatty acid chosen from stearic acid and palmitic acid, and mixtures thereof, and
(3) at least one base chosen from aminomethylpropanediol and alkali metal hydroxides, and
(4) at least one pullulan, and
(5) at least one polyol, and
(6) water; said composition containing less than 5.0% by weight of wax(es).

In yet another particularly preferred embodiment, the composition according to the invention comprises:
(1) at least one linear fatty acid monoester of formula (I):

R₁—O—R₂ (I)
wherein R₁ and R₂ are linear and saturated and have, independently of one another, a number of carbon atoms ranging from 20 to 24, with R₁ representing an acyl radical, and R₂ representing an alkyl radical,
(2) at least one fatty acid chosen from stearic acid and palmitic acid, and mixtures thereof, and
(3) at least one base chosen from aminomethylpropanediol and alkali metal hydroxides, and
(4) at least one pullulan, and
(5) at least one polyol chosen from 1,3-propanediol, glycerol and pentylene glycol, and mixtures thereof, and
(6) water; said composition containing less than 5.0% by weight of wax(es).

In yet another most particularly preferred embodiment, the composition according to the invention comprises:
(1) at least behenyl behenate as linear fatty acid monoester, and
(2) at least one fatty acid chosen from stearic acid and palmitic acid, and mixtures thereof, and
(3) at least aminomethylpropanediol as base, and
(4) at least one pullulan, and
(5) at least one polyol chosen from 1,3 propanediol and glycerol, and mixtures thereof,
(6) water; said composition containing less than 5.0% by weight of wax(es).

In another most particularly preferred embodiment, the composition according to the invention comprises:
(1) at least behenyl behenate as linear fatty acid monoester, and
(2) at least one fatty acid chosen from stearic acid and palmitic acid, and mixtures thereof, and
(3) at least one alkali metal hydroxide as base, and
(4) at least one pullulan, and
(5) at least one mixture of 1,3 propanediol and glycerol, and
(6) water; said composition containing less than 5.0% by weight of wax(es).

### Other Components

In addition to the abovementioned compounds, a composition according to the invention may of course comprise secondary ingredients.

### a) Waxes

Thus, a composition according to the invention may also comprise a wax.

However, with regard to the objectives targeted by the present invention, the compositions according to the invention comprise a reduced amount of wax of less than 5.0% by weight, or even less than 3.0% by weight of waxes, relative to the total weight of said waxes.

As specified in the preamble, for the purposes of the invention, the term “waxes” refers to lipophilic compounds, which are solid at ambient temperature (25° C.) and at atmospheric pressure (760 mmHg), with a reversible solid/liquid change of state, which have a melting point of greater than or equal to 40° C., which may be up to 120° C.

It is recalled that, for the purposes of the invention, the waxes to which the abovementioned amount limitation relates are distinct from those capable of being embodied by the component which is a linear fatty acid monoester (1) according to the invention and additional fatty alcohol components as defined below.

This limitation relates more particularly to waxes made up of complex mixtures which are notably described in Ullmann's Encyclopedia of Industrial Chemistry 2015, Wiley-VCH Verlag GmbH & Co. KGaA.

Such waxes may notably be natural, but may also be synthetic.

The term “natural” wax is intended to denote any wax which pre-exists in nature or which can be converted, extracted or purified from natural compounds which exist in nature.

Among natural waxes, mention may notably be made of waxes termed fossil waxes, including those of petroleum origin, such as ozokerite, pyropissite, macrocrystalline waxes, also known as paraffins—including crude or gatsch waxes, gatsch raffinates, de-oiled gatsch, soft waxes, semirefined waxes, filtered waxes, refined waxes—and microcrystalline waxes, termed microwaxes, including bright stock gatsch. The fossil waxes also contain lignite, also known as montan wax, or peat wax.

As natural waxes other than fossil waxes, mention may be made of animal and plant waxes.

As examples of plant waxes, mention may be made of carnauba wax, candelilla wax, ouricury wax, sugarcane wax, jojoba wax, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, mixtures thereof and derivatives thereof.

As examples of animal waxes, mention may be made of beeswax, Ghedda wax, shellac, Chinese wax, lanolin, also known as wool wax, mixtures thereof and derivatives thereof.

These waxes are generally multicomponent. For example, natural beeswax is composed of approximately 70% of esters for the majority of monoesters (of fatty acid and of fatty alcohol), but also of hydroxy esters, of diesters and triesters and esters of sterols, and also of long-chain linear hydrocarbons, of free acids and of free alcohols. For obvious reasons, the weight proportion of their ingredients and their degree of purity are difficult to guarantee from one production batch to another.

The term “synthetic” wax is intended to denote waxes of which the synthesis requires one or more chemical reactions performed by a human being.

Among the synthetic waxes, semi-synthetic waxes and totally synthetic waxes can be distinguished. Synthetic waxes may be waxes obtained via a Fischer-Tropsch process, constituted for example of paraffins with a number of carbon atoms ranging from 20 to 50 or waxes of polyolefins, for example homopolymers or copolymers of ethylene, of propene or butene, or even longer-chain α-olefins. The latter can be obtained by thermomechanical degradation of polyethylene plastic, by the Ziegler process, by high-pressure processes, or else via processes catalyzed by metallocene species. These waxes may be crystallizable, partially crystallizable or amorphous. The abovementioned synthetic waxes are generally apolar and can be chemically treated to obtain polar waxes, for example via one or more of the following reactions: air oxidation, grafting, esterification, neutralization with metal soaps, amidation, direct copolymerizations or addition reactions.

In this case also, their composition may be constituted of a mixture of ingredients since the fatty-chain lengths are not well defined, thus forming a mixture of compounds having different fatty-chain lengths and for which it is difficult for manufacturers to guarantee perfect reproducibility from one production batch to another.

Consequently, the compositions according to the invention advantageously comprise less than 3% by weight of waxes, notably of multicomponent natural or synthetic waxes, relative to the total weight of the composition.

For the purposes of the invention, a multicomponent wax denotes a wax constituted of a mixture of several ingredients, either such that it exists naturally like natural waxes, or such that it is formed during the process of industrial synthesis of these materials.

In a particularly preferred embodiment of the invention, the composition is free of these waxes, notably multicomponent natural or synthetic waxes.

As specified above, the preferred texturing compounds according to the invention are by contrast and advantageously synthetic, single-component compounds, which are thus available in a form purified to more than 99%, like compound (1) required according to the invention.

### b) Other Surfactants

The composition according to the invention may comprise surfactants other than that formed by the neutralization of a fatty acid having from 14 carbon atoms to less than 20 carbon atoms (2) with the base (3), as co-surfactants.

However, according to one preferred embodiment of the invention, the composition comprises less than 5.0% by weight, preferably less than 2.0% by weight, relative to the total weight of the composition, of nonionic surfactants, and in particular of nonionic surfactants with an HLB, measured at 25° C., of greater than or equal to 7.

The term “HLB” (Hydrophilic Lipophilic Balance) is well known to those skilled in the art, and denotes the hydrophilic-lipophilic balance of a surfactant determined at 25° C. in the Griffin sense. The term “hydrophilic-lipophilic balance (HLB)” means the equilibrium between the size and strength of the hydrophilic group and the size and strength of the lipophilic group of the surfactant. The HLB value according to Griffin is defined in J. Soc. Cosm. Chem. 1954 (volume 5), pages 249-256.

In a particularly preferred embodiment of the invention, the composition is free of nonionic surfactants with an HLB, measured at 25° C., of greater than or equal to 7.

The expression “composition devoid of nonionic surfactants with an HLB, measured at 25° C., of greater than or equal to 7” is intended to mean a composition comprising less than 1% by weight of nonionic surfactant with an HLB, measured at 25° C., of greater than or equal to 7, or even less than 0.5% by weight relative to the total weight of the composition, or even totally free of nonionic surfactant with an HLB of greater than or equal to 7.

### c) Liquid Fatty Phase

A composition according to the invention may also comprise a liquid fatty phase.

Such a liquid fatty phase is an organic phase that is liquid at ambient temperature (25° C.) and at atmospheric pressure (760 mmHg), non-aqueous and water-immiscible.

The liquid fatty phase may contain a nonvolatile oil chosen from polar oils and non-polar oils, and mixtures thereof.

A composition according to the invention may comprise from 1.0% to 20.0% by weight, from 2.0% to 12.0% by weight and preferentially from 2.0% to 8.0% by weight of nonvolatile oil, relative to the total weight of the composition.

A composition according to the invention preferably comprises less than 5.0% by weight and more preferentially less than 2.0% by weight of volatile oil(s), relative to the total weight of the composition. In one particularly preferred embodiment of the invention, the composition is free of volatile oils.

The term “volatile oil” is intended to mean to an oil that can evaporate on contact with the skin in less than one hour, at ambient temperature (25° C.) and atmospheric pressure (760 mmHg). More specifically, a volatile oil has an evaporation rate ranging from 0.01 to 200 mg/cm²/min.

### d) Colorant

A composition according to the invention, and notably those intended for makeup, generally comprises at least one colorant such as pulverulent materials, liposoluble dyes or water-soluble dyes.

The pulverulent colorants may be chosen from pigments and nacres.

The pigments may be white or colored, mineral and/or organic, and coated or uncoated. Among the mineral pigments that may be mentioned are titanium dioxide, optionally surface-treated, zirconium oxide, zinc oxide or cerium oxide, and also iron oxide, chromium oxide, manganese violet, ultramarine blue, chromium hydrate and ferric blue. Mention may be made, among the organic pigments, of carbon black, pigments of D & C type, and lakes based on cochineal carmine and on barium, strontium, calcium or aluminum.

The nacres may be chosen from white nacreous pigments such as mica coated with titanium or with bismuth oxychloride, colored nacreous pigments such as titanium mica with iron oxides, titanium mica notably with ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the composition according to the invention comprises a pulverulent colorant, preferably of pigment type, in particular metal oxides, especially titanium dioxides, iron oxides, and mixtures thereof; and more particularly iron oxides.

Preferably, said colorant is present in the composition in a content ranging from 2.0% to 25.0% by weight, preferably from 3.0% to 20.0% by weight, more particularly from 4.0% to 15.0% by weight, relative to the total weight of the composition.

### e) Cosmetic Active Agents

As cosmetic active agents that may be used in the compositions according to the invention, mention may be made notably of antioxidants, preserving agents, fragrances, neutralizers, cosmetic active agents, for instance emollients, vitamins and screening agents, in particular sunscreens, and mixtures thereof.

These additives can be present in the composition in a content ranging from 0.01% to 15.0% of the total weight of the composition.

Needless to say, those skilled in the art will take care to select the optional additional additives and/or the amount thereof such that the advantageous properties of the composition according to the invention are not, or are not substantially, adversely affected by the envisaged addition.

### f) Semicrystalline Polymer

A composition according to the invention may also comprise at least one semicrystalline polymer.

The composition according to the invention comprises preferably at least 2.0% by weight, more preferentially from 3.0% to 20.0% by weight, better still from 4.0% to 15.0% by weight, even better still from 5.0% to 15.0% by weight of semicrystalline polymer(s), relative to the total weight of the composition.

For the purposes of the invention, the term “polymers” means compounds including at least two repeating units, preferably at least three repeating units and more especially at least ten repeating units.

For the purposes of the invention, the term “semicrystalline polymer” means polymers including a crystallizable portion and an amorphous portion and having a first-order reversible change of phase temperature, in particular of melting (solid-liquid transition). The crystallizable portion is preferably a chain that is lateral (or a chain that is pendent) relative to the backbone.

Besides the crystallizable chains or blocks, the blocks of the polymers are amorphous.

For the purposes of the invention, the term “crystallizable chain or block” means a chain or block which, if it were alone, would change from the amorphous state to the crystalline state reversibly, depending on whether the temperature is above or below the melting point. For the purposes of the invention, a chain is a group of atoms, which is pendent or lateral relative to the polymer backbone.

When the crystallizable portion is a chain that is pendent relative to the backbone, the semicrystalline polymer may be a homopolymer or a copolymer.

Preferably, the semicrystalline polymer has an organic structure.

The term “organic compound” or “having an organic structure” refers to compounds containing carbon atoms and hydrogen atoms and optionally heteroatoms such as S, O, N or P, alone or in combination.

The semicrystalline polymer(s) according to the invention are solids at ambient temperature (25° C.) and atmospheric pressure (760 mmHg), the melting point of which is greater than or equal to 30° C.

The melting point values correspond to the melting point measured using a differential scanning calorimeter (D.S.C.) such as the calorimeter sold under the name DSC 30 by the company Mettler, with a temperature rise of 5° C. or 10° C. per minute (the melting point considered is the point corresponding to the temperature of the most endothermic peak in the thermogram).

According to a particular embodiment, the semicrystalline polymer(s) used in the composition of the invention have a melting point m.p. of less than 95° C., preferably less than 85° C. The semicrystalline polymer(s) may thus have a melting point m.p. ranging from 30 to 95° C. and preferably from 40 to 85° C. This melting point is preferably a first-order change of state temperature.

According to the invention, the semicrystalline polymers are advantageously soluble in the fatty phase, notably to at least 1.0% by weight, at a temperature above their melting point. Preferably, the polymer backbone of the semicrystalline polymers is soluble in the fatty phase at a temperature above their melting point.

Preferably, the crystallizable blocks or chains of the semicrystalline polymers represent at least 30% of the total weight of each polymer and better still at least 40%.

When the semicrystalline polymers of the invention are crystallizable block polymers they may be block or multiblock copolymers. They may be obtained by polymerizing a monomer bearing reactive (or ethylenic) double bonds or by polycondensation. When the polymers of the invention are polymers bearing crystallizable side chains, these polymers are advantageously in random or statistical form.

Preferably, the semicrystalline polymers of the invention are of synthetic origin.

According to a preferred embodiment, the semicrystalline polymer is chosen from homopolymers and copolymers including units resulting from the polymerization of one or more monomers bearing crystallizable hydrophobic side chain(s).

The semicrystalline polymers that may be used in the invention may be chosen in particular from homopolymers or copolymers, in particular those bearing at least one crystallizable side chain, such as those described in U.S. Pat. No. 5,156,911.

In a preferred embodiment, the crystallizable side chain(s) are hydrophobic.

These homopolymers or copolymers may result:
from the polymerization, in particular the free-radical polymerization, of one or more monomers containing double bond(s) which is (are) reactive, or which are ethylenic, with respect to a polymerization, namely a vinyl, (meth)acrylic or allylic group,
from the polycondensation of one or more monomers bearing co-reactive groups (carboxylic acid, sulfonic acid, alcohol, amine or isocyanate), for instance polyesters, polyurethanes, polyethers or polyureas.
   In general, the crystallizable units (chains or blocks) of the semicrystalline polymers according to the invention are derived from monomer(s) containing crystallizable block(s) or chain(s), used for manufacturing semicrystalline polymers. These polymers are preferably chosen notably from homopolymers and copolymers resulting from the polymerization of at least one monomer containing crystallizable chain(s) that may be represented by the formula below:

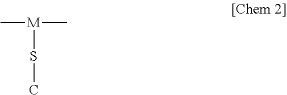

with M representing an atom of the polymer backbone, C representing a crystallizable group, and S representing a spacer, the crystallizable “—S—C” chains being hydrocarbon-based aliphatic or aromatic chains, including saturated or unsaturated hydrocarbon-based alkyl chains, which are for example C₁₀-C₄₀, preferably C₁₀-C₃₀. “C” notably represents a linear or branched or cyclic group (CH₂)ₙ, with n being an integer ranging from 10 to 40. Preferably, “C” is a linear group. Preferably, “S” and “C” are different.

When the crystallizable chains are hydrocarbon-based aliphatic chains, they comprise hydrocarbon-based alkyl chains containing at least 10 carbon atoms and not more than 40 carbon atoms and better still not more than 30 carbon atoms. They are notably aliphatic chains or alkyl chains containing at least 10 carbon atoms, and they are preferably C₁₀-C₄₀, preferably C₁₀-C₃₀, alkyl chains.

Preferably, the crystallizable chains are C₁₀-C₃₀ hydrocarbon-based aliphatic chains.

As examples of semicrystalline homopolymers or copolymers containing crystallizable chain(s) that are suitable for use in the invention, mention may be made of those resulting from the polymerization of one or more of the following monomers: saturated alkyl (meth)acrylates with the alkyl group being C₁₀-C₃₀, N-alkyl(meth)acrylamides with the alkyl group being C₁₀ to C₃₀, vinyl esters containing alkyl chains with the alkyl group being C₁₀ to C₃₀, vinyl ethers containing alkyl chains with the alkyl group being C₁₀ to C₃₀, C₁₀ to C₃₀ alpha-olefins, for instance octadecene, para-alkylstyrenes with an alkyl group including from 10 to 30 carbon atoms, and mixtures thereof.

When the polymers result from a polycondensation, the hydrocarbon-based crystallizable chains as defined above are borne by a monomer that may be a diacid, a diol, a diamine or a diisocyanate.

When the polymers that are subjects of the invention are copolymers, they additionally contain from 0% to 50% of groups Y which is a polar monomer, a nonpolar monomer or a mixture of the two.

When Y is a polar monomer, it is either a monomer bearing polyoxyalkylene groups (notably oxyethylene and/or oxypropylene groups), a hydroxyalkyl (meth)acrylate, for instance hydroxyethyl acrylate, (meth)acrylamide, an N-alkyl(meth)acrylamide, an N,N-dialkyl(meth)acrylamide, for instance N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a nonpolar monomer it may be an ester of the linear, branched or cyclic alkyl (meth)acrylate type, a vinyl ester, an alkyl vinyl ether, an α-olefin, styrene or styrene substituted with a C₁ to C₁₀ alkyl group, for instance α-methylstyrene, or a macromonomer of the polyorganosiloxane type containing vinyl unsaturation.

Preferably, the semicrystalline polymers containing one or more crystallizable side chain(s) are alkyl (meth)acrylate or alkyl(meth)acrylamide homopolymers with an alkyl group as defined above, and in particular of C₁₀-C₃₀, copolymers of these monomers with a hydrophilic monomer preferably of different nature from (meth)acrylic acid, for instance N-vinylpyrrolidone or hydroxyethyl (meth)acrylate, and mixtures thereof.

It is also possible to use the semicrystalline polymers obtained by copolymerization of behenyl acrylate and of acrylic acid or of N-vinylpyrrolidone, as described in U.S. Pat. No. 5,519,063.

Advantageously, the semicrystalline polymer(s) containing crystallizable side chain(s) have a weight-average molecular weight Mw ranging from 5000 g/mol to 1 000 000 g/mol, preferably from 10 000 g/mol to 800 000 g/mol, preferentially from 15 000 g/mol to 500 000 g/mol, and more preferably from 80 000 g/mol to 200 000 g/mol.

According to a particular embodiment of the invention, the semicrystalline polymer may be chosen from homopolymers and copolymers resulting from the polymerization of at least one monomer containing crystallizable side chain(s) chosen from saturated C₁₀ to C₃₀ alkyl (meth)acrylates, which may be represented by the following formula:

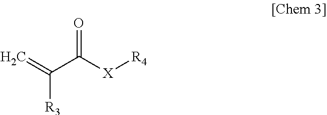

wherein R₃ is H or CH₃, R₄ represents a C₁₀ to C₃₀ alkyl group, and X represents O.

According to a more particular embodiment of the invention, the semicrystalline polymer results from the polymerization of monomers containing crystallizable side chain(s), chosen from saturated C₁₀ to C₃₀ alkyl (meth)acrylates.

The semicrystalline polymers comprising a crystallizable side chain may be chosen from copolymers resulting from the copolymerization of acrylic acid and C₁₀ to C₃₀ alkyl (meth)acrylate, notably such as those described in patent U.S. Pat. No. 5,156,911.

The semicrystalline polymers may notably be those described in examples 3, 4, 5, 7 and 9 of patent U.S. Pat. No. 5,156,911, and more particularly those obtained by the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of polyoctadecyl methacrylate in a 2.5/97.5 ratio.

As a particular example of a semicrystalline polymer that may be used in the composition according to the invention, mention may be made of the Intelimer® products from the company Landec described in the brochure “Intelimer® polymers”, Landec IP22 (Rev. 4-97). These polymers are in solid form at ambient temperature (25° C.). They bear crystallizable side chains. They are poly(C₁₀-C₃₀)alkyl acrylates, which are particularly suitable as semicrystalline polymers that may be included in a composition in accordance with the present invention.

According to a particularly preferred embodiment of the invention, the semicrystalline polymer(s) (5) according to the invention are derived from a monomer containing a crystallizable chain chosen from saturated C₁₀ to C₃₀ alkyl (meth)acrylates and more particularly from poly(stearyl acrylate)s, poly(behenyl acrylate)s, and mixtures thereof.

Preferably, the semicrystalline polymers that are suitable for use in the invention are notably poly(stearyl acrylate), in particular the product sold under the name Intelimer® IPA 13-1, from the company Air Products and Chemicals or Landec, which is a poly(stearyl acrylate) with a melting point equal to 49° C., or poly(behenyl acrylate), sold under the name Intelimer® IPA 13-6, from the company Air Products and Chemicals or Landec, which is a poly(behenyl acrylate) with a melting point equal to 65° C.

According to a particularly preferred embodiment, the semicrystalline polymer is at least one poly(behenyl acrylate).

Examples of homopolymers or copolymers suitable as semicrystalline polymers for the invention preferably comprise from 50% to 100% by weight of units resulting from the polymerization of one or more monomers bearing a crystallizable hydrophobic side chain.

### Fatty Alcohol

Preferentially, the compositions according to the invention also comprise at least one fatty alcohol. A composition can therefore comprise a single fatty alcohol according to the invention or several distinct fatty alcohols.

If several distinct fatty alcohols are present, they can be added separately during the preparation of the composition and the mixture thereof can then be formed in situ. They can also be used in the form of a mixture which is already commercially available and in which the weight proportion and the degree of purity of each of the fatty alcohols are controlled. In other words, the composition of these mixtures is faithfully reproducible, as opposed to mixtures of fatty alcohols generated via synthesis from starting materials derived from complex mixtures.

The fatty alcohol(s) are in particular chosen from linear or branched, saturated or unsaturated C₁₄-C₃₀, preferably C₁₄-C₂₄, and better still C₁₄-C₂₀, fatty alcohols.

The fatty alcohol(s) are in particular chosen from linear and saturated C₁₄-C₃₀ fatty alcohols, preferably linear and saturated C₁₄-C₂₄ fatty alcohols, and better still linear and saturated C₁₄-C₂₀ fatty alcohols.

According to a particularly preferred embodiment, the fatty alcohol is in the form of a mixture of several different fatty alcohols, and is preferably a mixture of several linear and saturated C₁₄-C₃₀, better still C₁₄-C₂₄, even better still C₁₄-C₂₀, fatty alcohols.

Preferably, the fatty alcohol according to the invention is chosen from cetyl alcohol (C₁₆), stearyl alcohol (C₁₈) and mixtures thereof (also known as “cetearyl alcohol”). Preferentially, the fatty alcohol according to the invention is a mixture of cetyl alcohol and stearyl alcohol. Such a mixture is notably sold under the name Lannette O OR/MB® by the company BASF.

According to a preferred embodiment, the fatty alcohol is solid at ambient temperature.

The fatty alcohol is present in the compositions of the invention in amounts ranging from 1.0% to 20.0% by weight, relative to the total weight of the composition, preferably from 2.0% to 15.0% by weight and even more particularly from 3.0% to 10.0% by weight, relative to the total weight of the composition.

### Physical Characteristics

### a) Solids Content

The composition according to the invention advantageously has a solids content of at least 42.0% by weight, and preferentially of at least 44.0% by weight, relative to the total weight of the composition, or even from 45.0% to 60.0% by weight, relative to the total weight of the composition.

For the purposes of the present invention, the term “solids content” denotes the content of nonvolatile matter.

The amount of solids content (abbreviated as SC) of a composition according to the invention is measured using a Halogen Moisture Analyzer HR 73® commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo®.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet weight of the sample, corresponding to its initial weight, and the dry weight of the sample, corresponding to its weight after halogen heating, are measured using a precision balance.

The experimental error associated with the measurement is of the order of plus or minus 2%.

The solids content is calculated in the following manner:

$$Solids Content ( expressed in \% by weight = 100 \times \frac{Dry weight}{Wet weight} \qquad [ Math 1 ]$$

### b) Viscosity

A composition according to the invention is advantageously creamy at an ambient temperature of 25° C.

Preferably, the viscosity of the compositions according to the invention ranges from 2.0 to 40.0 Pa·s, or even preferably from 2.5 to 35.0 Pa·s, more particularly from 3.0 to 30.0 Pa·s, measured at the ambient temperature of 20° C. by means of a Rheomat RM100® with a spindle 4 and with a shear rate of 200 rpm and a shear time of 10 min.

Such a viscosity is particularly advantageous since it is the most suitable for the device for applying mascara and since it enables easy use for the consumer for a charging result.

The composition may be manufactured via the known processes generally used in the cosmetics field.

The composition used according to the invention may be a makeup composition, a makeup base, notably for keratin fibers, or base coat, a composition to be applied onto makeup, also known as topcoat, or else a composition for treating keratin fibers.

More especially, the composition according to the invention is a mascara.

Such compositions are notably prepared according to the general knowledge of those skilled in the art.

### Packaging and Application Assembly or Kit

The present invention also relates to an assembly, or kit, for packaging and applying a cosmetic composition for coating keratin fibers, comprising:
a device for packaging said cosmetic composition for coating keratin fibers as described above,
an applicator of said composition.

Said applicator may be integrally attached to a gripping member forming a cap for said packaging device. In other words, said applicator may be mounted in a removable position on said device between a closed position and an open position of a dispensing aperture of the device for packaging said composition.

An assembly for coating keratin fibers suitable for the invention may comprise an applicator configured for applying said cosmetic composition for coating keratin fibers and, where appropriate, a packaging device suitable for receiving said composition.

### Applicator

The applicator comprises means for smoothing and/or separating keratin fibers, such as the eyelashes or the eyebrows, notably in the form of teeth, bristles, spikes or other reliefs.

The applicator is arranged to apply the composition to the eyelashes or the eyebrows, and may comprise, for example, a brush or a comb.

The applicator may also be used for finishing of the makeup, over a region of the eyelashes or eyebrows that is made up or laden with the composition.

The brush may comprise a twisted core and bristles held between the turns of the core, or may be made in yet another way.

The comb is, for example, produced from a single part by molding of a plastic.

In certain exemplary embodiments, the application member is mounted at the end of a wand, which wand may be flexible, which may contribute to improving the comfort during application.

### Packaging Device

The packaging device comprises a container intended for housing the composition for coating keratin fibers. This composition may then be withdrawn from the container by immersing the applicator therein.

This applicator may be firmly attached to a member for closing the container. This closing member may form a member for gripping the applicator. This gripping member may form a cap to be removably mounted on said container by any suitable means, such as by screwing, click-fastening, coupling, etc. Such a container may thus reversibly house said applicator.

This container may be optionally equipped with a wiper suitable for removing surplus product taken up by the applicator.

A process for applying the composition according to the invention to the eyelashes or the eyebrows may also include the following steps:
forming a deposit of the cosmetic composition on the eyelashes or the eyebrows,
leaving the deposit on the eyelashes all the eyebrows, it being possible for the deposit to dry.

It should be noted that, according to another embodiment, the applicator may form a product container. In such a case, a container may, for example, be provided in the gripping member and an internal channel can internally connect this gripping member to the application members in relief.

Finally, it should be noted that the packaging and application assembly may be in the form of a kit, it being possible for the applicator and the packaging device to be housed separately in the same packaging article.

The expressions “between . . . and . . . ”, “of between . . . and . . . ” and “ranging from . . . to . . . ” should be understood as meaning limits included, unless otherwise specified.

In the description and the examples, the percentages are weight percentages, unless otherwise indicated. The percentages are thus expressed on a weight basis relative to the total weight of the composition. The ingredients are mixed in the order and under the conditions which are readily determined by those skilled in the art.

The invention will now be described by means of examples which are present for purely illustrative purposes and should not be interpreted as examples that limit the invention.

### COMPOSITION EXAMPLES

### Example 1 (Invention), Example 2 (Outside the Invention)

The following mascara examples 1 and 2 are prepared.

TABLE 1

| | | Ex 1 | Ex 2 |
|---|---|---|---|
| | | according | Outside |
| | | to the | the |
| Phase | Ingredients (INCI name) | invention | invention |
| A | Cetearyl alcohol | 4.0 | 4.0 |
| | Lanette O OR/MB ® from BASF | | |
| | Behenyl behenate (C44) | 15.0 | 15.0 |
| | (fatty acid monoester (1)) | | |
| | Kester Wax K-72 ® from Koster | | |
| | Keunen | | |
| | Poly(C₁₀-C₃₀ alkyl acrylate) | 6.0 | 6.0 |
| | Intelimer IPA 13-6 Polymer ® | | |
| | Air Products and Chemicals | | |
| | Stearic acid | 6.0 | 6.0 |
| | Stearic Acid 1850 ® from Southern | | |
| | Acids | | |
| | Iron oxides | 8.0 | 8.0 |
| | (fatty acid (2)) | | |
| | Sunpuro Black Iron Oxide | | |
| | C33-7001 ® from Sun | | |
| B | Pullulan (4) | 4.0 | 0.0 |
| | 1,3-Propanediol (polyol (5)) | 3.0 | 3.0 |
| | Activonol-3 ® from Activon | | |
| | Glycerol (polyol (5)) | 2.0 | 2.0 |
| | Glycerin USP Kosher ® from Cargill | | |
| | Phenoxyethanol | 0.6 | 0.6 |
| | Chlorphenesin | 0.3 | 0.3 |
| | Aminomethyl propanediol (base (3)) | 1.0 | 1.0 |
| | AMPD Ultra PC ® from Angus | | |
| | (Dow Corning) | | |
| | Water (6) | q.s. 100 | q.s. 100 |

### Protocol for Preparing the Examples 1 and 2

### Preparation of Phase A

The starting materials were carefully weighed out beforehand using a balance (precision=0.01 g). The ingredients of phase A were melted in a jacketed heating pan in which circulates an oil of which the temperature is controlled by means of a thermostatically-controlled oil bath. The set point temperature was set at 90° C. After total melting, the pigment was introduced with stirring using a Rayneri® blender. Stirring was maintained until a homogeneous preparation was obtained.

### Preparation of Phase B

The water was preheated in an electric kettle to 95° C. The cationic polymer and the anionic polymer were mixed with the hot water and then the preserving agents and the base were introduced into the water in a beaker at a temperature of 80° C. with stirring using a Rayneri® blender.

### Emulsification of Phases A and B

Phase B was poured into phase A with stirring for 5 minutes at 90° C. using a Rayneri blender. Phase A+B was then cooled to ambient temperature with stirring.

### Packaging of the Formulation

The mascara formula obtained was then transferred into a hermetically closed jar in order to prevent drying of the formula in contact with the air.

### Comparative Tests

a) Appearance of the Mascaras

After 24 h at ambient temperature (25° C.) and atmospheric pressure, the macroscopic appearance of the sample and also the state of dispersion of the fatty substances and of the pigments thereof were verified using an optical microscope. Likewise, the viscosity of the formula is measured.

The appearance of the composition was observed. The following notations A, B and C were given:
A: Homogeneous, thick, black and smooth composition.
B: Homogeneous, thick, black and mat composition.
C: Heterogeneous composition with 2 liquid phases.

b) Mechanical Properties of the Deposit of the Formulation on the Eyelashes

In order to evaluate the staying power of the mascara deposit with respect to rubbing, the formula was packaged in a mascara heating vessel and the final product was applied to a false-eyelash test specimen and left to dry for 4 hours. The resistance of the mascara deposit on the false-eyelash test specimen was then challenged using a brush bearing no mascara. For that, the bare brush was used to rub the deposit (3×10 passages) and the amount of black deposit lost by flaking of the deposit was collected on a white support and then evaluated.

The appearance of the supports after rubbing of the mascara deposit was observed. The following notations A, B, C and D were given:
A: no flaking is observed;
B: slight flaking is observed;
C: flaking is observed;
D: considerable flaking is observed.

A support comprising strong flaking generates a crumbly mascara deposit and poor resistance to rubbing. Conversely, a support comprising no flaking generates a non-crumbly mascara deposit and good resistance to rubbing.

The results obtained are shown in the table below.

TABLE 2

| | Ex 1 | Ex 2 |
|---|---|---|
| | according to | Outside the |
| Tests carried out | the invention | invention |
| Appearance after 24 hours | A | A |
| Viscosity at 25° C. with a Rheomat | 4.5 | 4.1 |
| RM100 ® (Pa.s) | | |
| Observation of flaking or | A | D |
| absence of flaking | | |

It was observed that example 1 according to the invention, after 24 hours, is homogeneous, thick, black and smooth. It exhibits no flaking and has good resistance to rubbing.

1. A composition comprising, in a physiologically acceptable medium:
(1) at least one linear fatty acid monoester of formula (I):
R₁—O—R₂ (I)
wherein Ri and R₂ are linear and saturated and have, independently of one another, a number of carbon atoms greater than or equal to 20, with R₁ representing an acyl radical, and R₂ representing an alkyl radical,
(2) at least one fatty acid having from 14 carbon atoms to less than 20 carbon atoms,
(3) at least one base capable of at least partially neutralizing said fatty acid,
(4) at least one pullulan,
(5) at least one polyol, and
(6) water;
said composition containing less than 5.0% by weight of wax(es), relative to the total weight of said composition.

2. The composition as claimed in claim 1, containing less than 3.0% by weight of wax(es), relative to the total weight of said composition.

3. The composition as claimed in claim 1, wherein the at least one linear fatty acid monoester of formula (1) is chosen from arachidyl arachidate, behenyl behenate, and mixtures thereof.

4. The composition as claimed in claim 1, comprising at least 5.0% by weight of linear fatty acid monoester(s) of formula (1) relative to the total weight of the composition.

5. The composition as claimed in claim 1, wherein the fatty acid(s) is (are) chosen from linear fatty acids, saturated fatty acids, and mixtures thereof.

6. The composition as claimed in claim 1, wherein the fatty acid(s) is (are) chosen from stearic acid, palmitic acid, and mixtures thereof.

7. The composition as claimed in claim 1, comprising at least 3.0% by weight of fatty acid(s), relative to the total weight of the composition.

8. The composition as claimed in claim 1, wherein the at least one base is at least one inorganic base.

9. The composition as claimed in claim 1, wherein the at least one base is at least one organic base.

10. The composition as claimed in claim 1, comprising at least 0.1% by weight relative to the total weight of the composition of the at least one base.

11. The composition as claimed in claim 1, wherein the at least one pullulan is present in a content ranging from 1% to 7% by weight relative to the total weight of the composition.

12. The composition as claimed in claim 1, wherein the at least one polyol is chosen from 1,3-propanediol, glycerol, pentylene glycol, and mixtures thereof.

13. The composition as claimed in claim 1, wherein the polyol(s) is/are present in a total content ranging from 5% to 10% relative to the total weight of said composition.

14. The composition as claimed in claim 1, comprising at least 30.0% by weight of water, relative to the total weight of the composition.

15. The composition as claimed in claim 1, also comprising at least one colorant.

16. The composition as claimed in claim 15, wherein the at least one colorant is chosen from metal oxides.

17. The composition as claimed in claim 1, also comprising at least one semicrystalline polymer.

18. The composition as claimed in claim 1, also comprising at least one fatty alcohol.

19. The composition as claimed in claim 1, in the form of an eyelash product, an eyebrow product or a product for the contour of the eyes.

20. An assembly, or kit, for packaging and applying a cosmetic composition for coating keratin fibers, comprising:
a device for packaging said cosmetic composition for coating keratin fibers as defined in claim 1;
an applicator of said composition.

21. A process for coating keratin materials comprising applying to said keratin materials at least one composition as defined in claim 1.
